(19) United States
(12) Patent Application Publication
(10) Pub. No.: US 2014/0141048 A1
(43) Pub. Date: May 22, 2014

(54) IONIC HYDROPHILIC POLYMER COATINGS FOR USE IN MEDICAL DEVICES

(71) Applicant: Boston Scientific Scimed, Inc., Maple Grove, MN (US)

(72) Inventors: David Rolf, Eden Prairie, MN (US); Steven L Kangas, Woodbury, MN (US)

(73) Assignee: Boston Scientific Scimed, Inc., Maple Grove, MN (US)

(21) Appl. No.: 14/084,821

(22) Filed: Nov. 20, 2013

Related U.S. Application Data
(60) Provisional application No. 61/728,919, filed on Nov. 21, 2012.

(51) Int. Cl. A61L 29/16

(52) U.S. Cl. CPC A61L 29/16
USPC 424/400; 514/56

(57) ABSTRACT

According to one aspect of the disclosure, medical devices are provided which have a negatively charged surface and a lubricous hydrophilic coating comprising a sulf(on)ated species disposed on the negatively charged surface. In various embodiments, the sulf(on)ated species is ionically crosslinked with itself and with the negatively charged species by a multivalent cationic species. In other aspects, medical devices are provided which have a polymeric surface and a lubricous hydrophilic layer comprising a covalently crosslinked sulf(on)ated species disposed on the surface. Still other aspects of the invention pertain to methods of forming such devices and methods of using such devices.

### STATEMENT OF RELATED APPLICATION

This application claims the benefit of U.S. Ser. No. 61/728,919, filed Nov. 21, 2012 and entitled: “IONIC HYDROPHILIC POLYMER COATINGS FOR USE IN MEDICAL DEVICES,” which is hereby incorporated by reference in its entirety.

### BACKGROUND

Hydrophilic coatings are coatings that exhibit strong chemical interactions with water, for example, by participating in hydrogen bonding with surrounding water molecules. In various instances, hydrophilic coatings are ionic, which further facilitates aqueous interactions. Hydrogel materials are capable of being readily wetted upon exposure to water and frequently form lubricious surfaces.

When employed in medical devices such as insertable guidewires and catheters, low coefficients of friction exhibited by hydrogel coatings can reduce the insertion force associated with such devices, allowing them to traverse body lumens more easily, while avoiding possible puncture damage and reducing abrasion between the device surfaces and the body lumens. Devices of this type, however, can experience significant shear stresses during use which can lead to particulate release due to fragmentation of the coating. Moreover, in some devices, particulate release can arise from the release and precipitation of chemical species that are present within materials that are used to form the devices. These and/or other issues in the medical device art are addressed by the hydrophilic polymer coatings of the present invention.

### SUMMARY OF THE INVENTION

According to one aspect of the disclosure, medical devices are provided which have a negatively charged surface and a lubricous hydrophilic coating comprising a sulf(on)ated species disposed on the negatively charged surface. In various embodiments, the sulf(on)ated species is ionically crosslinked with itself and with the negatively charged species by a multivalent cationic species.

In other aspects, medical devices are provided which have a polymeric surface and a lubricous hydrophilic layer comprising a covalently crosslinked sulf(on)ated species disposed on the surface.

Still other aspects of the invention pertain to methods of forming such devices and methods of using such devices.

These and other aspects, as well as various embodiments and advantages of the present invention will become immediately apparent to those of ordinary skill in the art upon review of the Detailed Description and claims to follow.

### BRIEF DESCRIPTION OF THE DRAWINGS

FIG. 1 is a schematic illustration of a process for grafting a polymer from a substrate surface, in accordance with the prior art.

FIG. 2 is a schematic illustration of an ionically crosslinked hydrophilic coating, in accordance with an embodiment of the present invention.

FIGS. 3-6 are schematic illustrations of processes for forming ionically crosslinked hydrophilic coatings, in accordance with various embodiments of the present invention.

### DETAILED DESCRIPTION OF THE INVENTION

A more complete understanding of the present invention is available by reference to the following detailed description of various aspects and embodiments of the invention. The detailed description of the invention which follows is intended to illustrate but not limit the invention. The scope of the invention is defined by any appended claims.

In accordance with one aspect, the present disclosure is directed to hydrophilic coatings for medical devices. As discussed further below, the hydrophilic coatings of the present disclosure are applicable to a wide variety of medical devices having a wide variety of surface materials, including organic and inorganic surface materials. As discussed further below, the hydrophilic coatings of the present disclosure may exhibit one or more of the following advantages, among others: (a) enhanced lubricity, (b) reduced particulate generation and (c) the ability to be readily bioabsorbed in the event that the coating materials become dislodged from the medical device.

Preferred hydrophilic coatings for use in accordance with the present disclosure are formed from materials that are sulfated, sulfonated or both. As used herein a “sulfonated” species is a species containing one or more —SO₃⁻Z⁺ groups (referred to herein as “sufonate groups’), where Z⁺ is a monovalent cationic entity such as H⁺, Li⁺, Na⁺, K⁺, etc. As used herein, a “sulfated” species is a species containing one or more —OSO₃⁻Z⁺ groups (referred to herein as “sulfate groups’). For convenience, species that are sulfonated, sulfated or both are collectively referred to herein as “sulfated/sulfonated” species or “sulf(on)ated” species.

Sulf(on)ated species suitable for forming hydrogel coatings in accordance with the present disclosure may be, for example, natural or synthetic, and they may be in the form of polymers or small-molecules.

In some embodiments, polymeric sulf(on)ated biomaterials are used in the formation of the hydrogel coatings. For example, sulf(on)ated polysaccharides such as glycosaminoglycans (GAG's) may be employed in the present disclosure. GAG's are ionic in nature and are comprised of repeat sugar monomer units with variability in sulf(on)ation at various locations on the monomers. These materials are found widely dispersed in nearly all mammals, including humans. Specific examples of GAG's include chondroitin sulfate, dermatan sulfate, keratan sulfate, and heparin. GAG's for use in the present disclosure typically range from 5,000 to 100,000 Daltons (e.g., 5,000 to 10,000 to 20,000 to 25,000 to 50,000 to 75,000 to 100,000 Daltons) in molecular weight, more typically 5,000 to 20,000 Daltons. Certain GAG's, such as dermatan sulfate and heparin are antithrombotic in nature, making them particularly useful, for example, in blood-contacting medical devices. Blends of two or more GAG's may also be employed. For example, a blend of heparin and one or more additional GAG's may allow the use of very small amounts of heparin, which an expensive and potent molecule, to be employed. For instance, one unit of heparin (i.e., a “Howell Unit”) is an amount approximately equivalent to 0.002 mg of pure heparin, which is the quantity required to keep 1 mL of cat's blood fluid for 24 hours at 0° C.

In other embodiments, sulf(on)ated small-molecule materials may be used in the present disclosure. Examples include sulf(on)ated small-molecule materials such as sulf(on)ated monosaccharides and sulf(on)ated oligosaccharides (defined herein as having between two and ten sugar units and thus including disaccharides, trisaccharides, tetrasaccharides, pentasaccharides, hexasaccharides, and so forth). Specific examples include sulf(on)ated glucose, sulf(on)ated fructose, sulf(on)ated galactose, sulf(on)ated lactose and sulf(on)ated sucrose. Typically, the saccharides will contain two, three, four, five, six, seven, eight or more sulfate and/or sulfonate sites. One example of a known sulfated saccharide is sucrose octasulfate, which is known as sucralfate when in hydrous basic aluminum salt form. Bulk sources of sodium or potassium salts of sucrose octasulfate are available.

Blends of sulf(on)ated small-molecule materials mixed with GAG's may be used to modify the properties of the coating.

In certain embodiments, sulf(on)ated monomers and synthetic sulf(on)ated polymers formed from sulf(on)ated monomers may be used in the present disclosure. Specific examples include sulfonic-acid-based monomers and their salts, for example, vinyl sulfonic acid, styrene sulfonic acid, vinyl toluene sulfonic acid, (meth)allyl sulfonic acid, (meth)allyloxybenzene sulfonic acid, 2-hydroxy-3-methacryloxypropyl sulfonic acid, and 2-acrylamido-2-methyl propane sulfonic acid (AMPS), among others, along with salts thereof (e.g., lithium, sodium, potassium, etc.). Synthetic polymers formed from each of these monomers and combinations of these monomers may be employed. In certain embodiments, additional non-sulfonic-acid-based monomers may be employed. For instance, in one specific example, poly(4 styrene sulfonic acid-co-maleic acid) and salts thereof may be employed.

By virtue of their high negative (anionic) charge the preceding materials are very hydrophilic and can be used to form lubricious, bioerodible coatings.

In various embodiments, covalent and/or ionic crosslinking mechanisms may be employed to create coatings having a wide range of biostabilities.

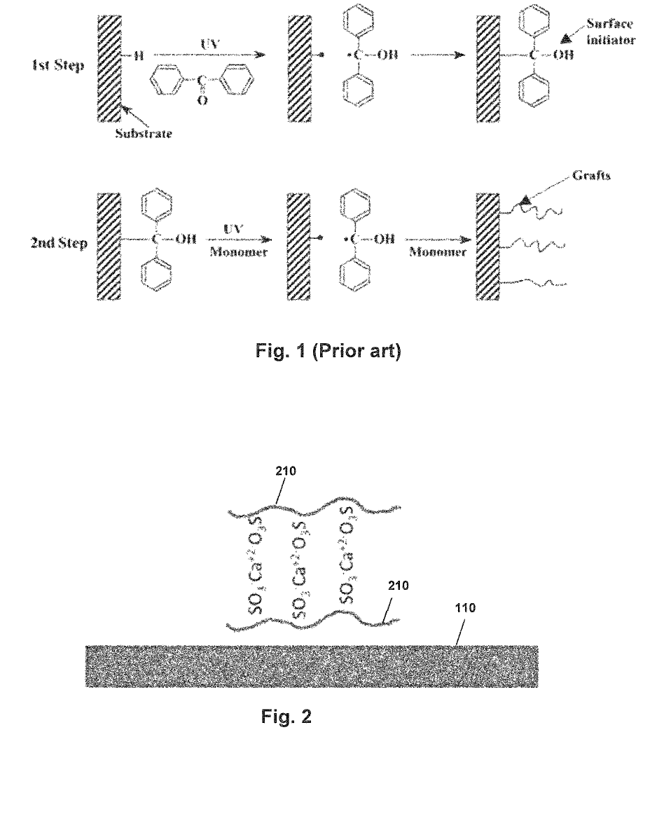

In some embodiments, a sulf(on)ated material, for instance, a natural or synthetic sulf(on)ated polymer such as those described above, among others, may be ionically crosslinked using multivalent metal cations (e.g., Mg²⁺, Ca²⁺, Sr²⁺, Ba²⁺, Fe²⁺, Al³⁺, Zn²⁺, etc.). This is shown schematically in FIG. 2 which shows two sulf(on)ated polymer molecules 210 (e.g., a GAG molecule, among numerous other possibilities) that are ionically crosslinked with multivalent metal cations (Ca²⁺). In one specific procedure, a solution of a multivalent metal salt (e.g., Ca(OH)₂, etc.) is applied to a medical device substrate 110 and dried. Then an aqueous solution of GAG polymer molecules 210 is applied over the multivalent metal salt, resulting in an ionic crosslinking of the GAG polymer molecules 210. (In another specific embodiment, the GAG is applied first, followed by application of the multivalent metal salt.) On exposure to physiologic solutions for sufficiently long enough periods, the ionically crosslinked GAG molecules 210 will disperse, for example, due to ion exchange of the multivalent ions with monovalent ions in body fluids such as blood (e.g., Na+, K+, etc.).

In other embodiments, a sulf(on)ated material, for instance, a natural or synthetic sulf(on)ated polymer such as those described above, among others, may be crosslinked using a suitable covalent crosslinking agent. In certain instances, crosslinking agents are selected which create bonds that are readily broken in physiologic solutions (e.g., due to hydrolysis, etc.)

Examples of suitable organic crosslinking agents include ester crosslinking agents, for instance, (a) orthoester crosslinking agents and (b) thiols (i.e., R—SH, where R is an organic radical), which can be reacted with carboxyl groups that may be present in sulf(on)ated materials (e.g., GAG's, etc.) to form thioesters, among other possibilities.

Orthoester crosslinking agents include non-cyclic orthoesters such as those of the general formula RC(OR′)3, where R is H or an organic radical (e.g., an alkyl group) and R′ is an organic radical (e.g., an alkyl group). Specific examples include triethyl orthoformate,

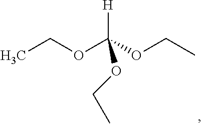

trimethyl orthoformate, trimethyl orthoacetate and triethyl orthoacetate, among others. Additional examples of orthoester crosslinking agents include bicycle-orthoesters and spiro-orthoesters. Orthoesters are capable of covalently crosslinking with alcohol and/or amine groups that may be present in sulf(on)ated materials (e.g., GAG's, etc.). In a specific procedure an aqueous solution of a sulf(on)ated material that contains alcohol and/or amine groups (e.g., a GAG, etc.) is applied to a medical device surface and dried. In a subsequent step, an anhydrous solution containing an orthoester (e.g., triethyl orthoformate) is applied to the dried sulf(on)ated material and heated to dry and crosslink the materials. On exposure to physiologic solutions for sufficiently long periods, these coatings will break down and disperse.

Various other crosslinking chemistries are known in the art that are suitable for crosslinking sulf(on)ated species that contain alcohol, amine, carboxylate and/or sulfate groups, all of which are present in GAG's.

In various additional embodiments, the medical device substrate surface is modified to have a negative charge, which can be used to enhance the adhesion of the coatings to the substrate.

In some embodiments, small-molecule or polymeric sulf(on)ated species may be covalently secured to the medical device surface using various techniques.

For instance, benzophenone and its derivatives may be used for surface grafting, which may be conducted in accordance with the scheme shown in FIG. 1. See Ma, H. M. et al., “A novel sequential photoinduced living graft polymerization,” Macromolecules, 33, 331-335 (2000). Without wishing to be bound by theory, it is believed that the surface grafting proceeds as follows: In a 1st step benzophenone is applied to a substrate to be modified, and the substrate exposed to UV radiation. The benzophenone absorbs this radiation, and facilitates the abstraction of hydrogen atoms from the surface of the substrate. Surface grafted benzophenone is formed by the recombination of the radicals generated from benzophenone and the radicals created on the substrate surface. Excess benzophenone that is unattached after surface grafting may then be washed away using a suitable solvent. In a 2nd step, the substrate with surface grafted benzophenone initiator groups may be exposed to UV radiation in the presence of monomers. The UV light cleaves the carbon-carbon bond of the surface grafted initiator species to form surface radicals and benzophenone radicals. Monomers are then able to react with the surface radicals, allowing polymer chains to be grafted from the substrate.

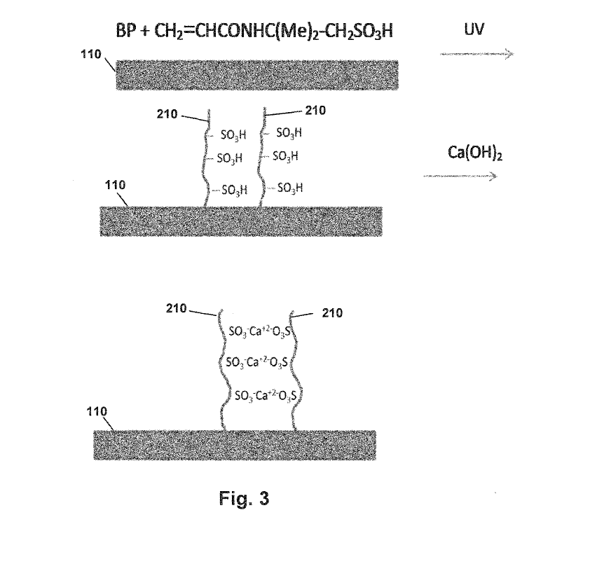

In one specific embodiment, a grafted surface initiator is used to polymerize a sulf(on)ated polymer at the surface of a medical device substrate using a suitable sulf(on)ated monomer. Referring now to FIG. 3, a UV surface initiator (e.g., surface grafted benzophenone (BP)) is used to polymerize a sulf(on)ated monomer (e.g., AMPS, CH₂═CHCONHC(Me)₂CH₂SO₃H), thereby forming a sulf(on)ated polymer 210 (e.g., polyAMPS) at the surface of a medical device substrate 110. If desired, the grafted sulf(on)ated polymer 210 can be ionically crosslinked using a suitable multivalent metal salt (e.g., Ca(OH)₂, etc.) as shown. Although not shown, an additional sulfonated material (e.g., GAG, etc.) can also be ionically crosslinked with the grafted sulfonated polymer 210. In one specific procedure, a solution of a multivalent metal salt (e.g., Ca(OH)₂, etc.) is applied to the grafted sulf(on)ated polymer 210. Subsequently, an aqueous GAG solution is applied, which is ionically crosslinked to itself and to the underlying grafted sulf(on)ated polymer 210.

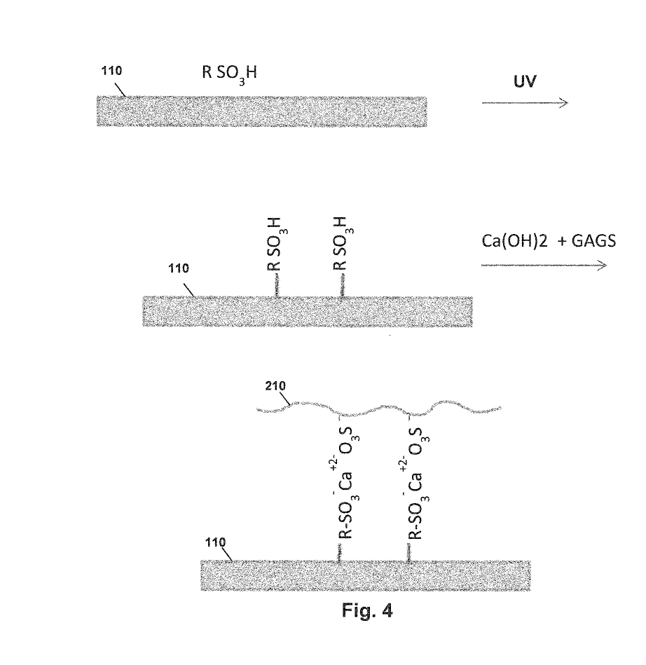

In other embodiments, a sulfated polymer or other sulfated species is directly attached to the substrate surface. For instance, as shown in FIG. 4, a sulf(on)ated species, RSO₃H, where R is an organic radical, may be attached to a substrate 210. In one example, the sulf(on)ated species may be a sulf(on)ated benzophenone derivative such as 5-benzoyl-4-hydroxy-2-methoxybenzenesulfonic acid,

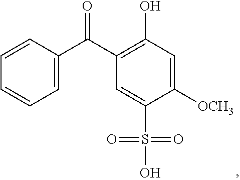 [SMILES: COc1cc(O)c(C(=O)c2ccccc2)cc1S(=O)(=O)O]

which can be grafted to the surface using a UV-based procedure analogous to that used to surface-graft benzophenone (discussed above). The surface-grafted sulf(on)ated species may then be ionically crosslinked to an additional sulf(on)ated species, for instance, a natural or synthetic sulf(on)ated polymer 210, using multivalent metal cations. In one specific procedure, a solution of a multivalent metal salt (e.g., Ca(OH)₂, etc.) is applied to the medical device substrate 110 with immobilized sulfonic species and dried, followed by application of an aqueous solution of GAG polymer molecules 210 to the multivalent metal salt, resulting in an ionic crosslinking between the surface-grafted sulf(on)ated species and the GAG polymer molecules 210.

Although sulfonated species are grafted to the medical device surface in FIGS. 3 and 4, anionic species other than sulf(on)ated species may be employed including carboxylate and phosphate species. For instance, in one specific example, a carboxylate monomer such as acrylic acid or methacrylic acid may be surface polymerized in a scheme analogous to the first step shown in FIG. 3, or a carboxylated benzophenone derivative may be surface attached in a scheme analogous to the first step shown in FIG. 4.

Other techniques which may be used to attach anionic species to a medical device surface are based on plasma treatment process. In one specific example, a carboxylated surface may be formed using a plasma treatment process in which a gas such as carbon monoxide (CO), carbon dioxide (CO₂), or oxygen (O₂) is used to functionalize a substrate surface with carboxyl groups. In another example, argon plasma treatment may be employed to create sulfate and carboxylate groups on substrate surfaces. See, e.g., J. P. Lens et al., “Preparation of heparin-like surfaces by introducing sulfate and carboxylate groups on poly(ethylene) using an argon plasma treatment,” J. Biomater. Sci. Polymer Edn., vol. 9, pp. 357-373, 1998.

Moreover, while covalently grafted anionic species are exemplified in FIGS. 3 and 4, anionic species may be held on the surface by other mechanisms including, cohesive mechanisms.

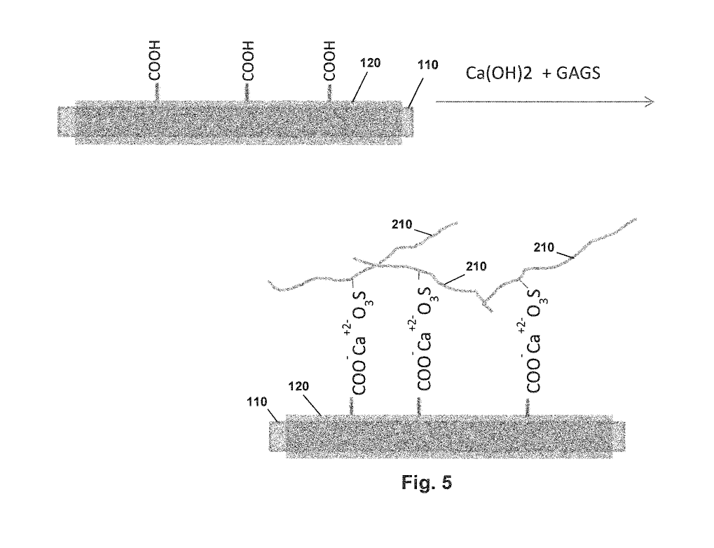

For instance, as schematically shown in FIG. 5, a coating of an anion-containing species (a coating of a carboxyl-containing species 120 is shown) may be provided on a medical device substrate 110. For example, a non-hydrogel carboxyl containing polymer, for instance, an acrylic acid copolymer such as acrylic acid-ethylene block copolymer or a non-hydrogel sulf(on)ate containing polymer such as polystyrene sulfonate, polyurethane sulfonate or poly(styrene-b-isobutylene-b-styrene) sulfonate, may be applied as a coating using a suitable thermoplastic or solvent-based process. This coating 120 may then be ionically crosslinked to a sulf(on)ated species 210 (e.g., a natural or synthetic sulf(on)ated polymer such as those described above, among others) using multivalent metal cations (e.g., Ca⁺², etc.). In one specific procedure, a solution of a multivalent metal salt (e.g., Ca(OH)₂, etc.) is applied to the carboxyl coating 120. Subsequently, an aqueous GAG solution is applied, which is ionically crosslinked to the underlying carboxyl coating 120.

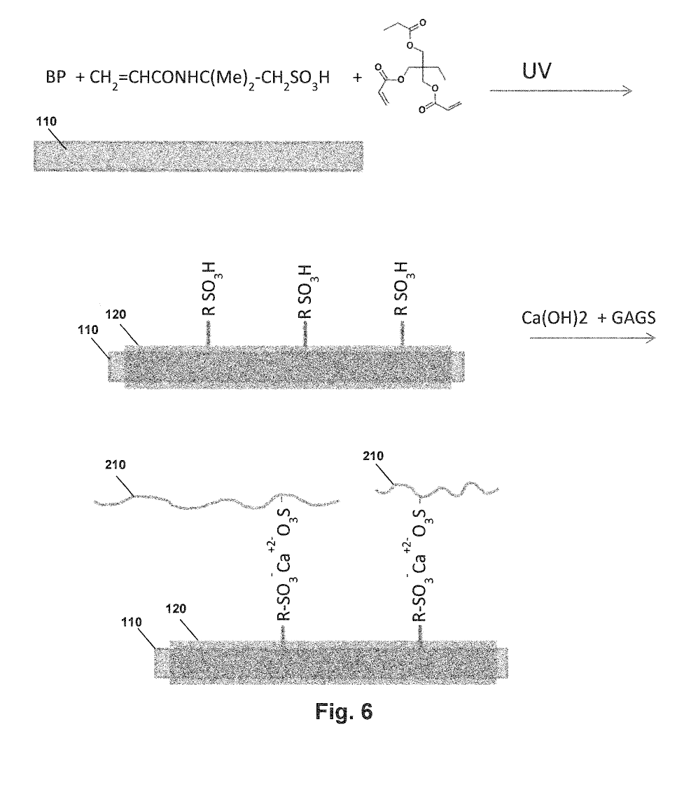

In still other embodiments, a coating of a covalently crosslinked anionic polymer may be provided. For example, referring to FIG. 6, (a) an initiator, for instance, benzophenone (BP), among others, (b) a monofunctional anionic monomer, for example, a sulf(on)ated monomer such as CH₂═CHCONHC(Me)₂CH₂SO₃H (AMPS), among others, and (c) a multifunctional monomer, for instance, neopentyl glycol diacrylate or trimethylolpropane triacrylate (TMPTA), among others, can be coated onto a medical device substrate 110 and UV cured to yield a crosslinked sulf(on)ated coating 120. This coating 120 may then be ionically crosslinked to a sulf(on)ated species 210 (e.g., a natural or synthetic sulf(on)ated polymer such as those described above, among others) using multivalent metal cations (e.g., Ca⁺², etc.). In one specific procedure, a solution of a multivalent metal salt (e.g., Ca(OH)₂) is applied to the crosslinked sulf(on)ated coating 120. Subsequently, an aqueous GAG solution is applied, which is ionically crosslinked to the underlying coating 120.

As noted above, the hydrophilic coatings of the present disclosure are applicable to a wide variety of medical devices having a wide variety of surface materials.

Medical devices to which coatings in accordance with the present disclosure may be applied include implantable or insertable medical devices which may be selected, for example, from wire interventional devices such as guidewires, diagnostic devices such as pressure wires, catheters including urological catheters and vascular catheters such as balloon catheters and various central venous catheters, balloons, vascular access ports, dialysis ports, stents (including coronary vascular stents, peripheral vascular stents, cerebral, urethral, ureteral, biliary, tracheal, gastrointestinal and esophageal stents), stent grafts, vascular grafts, abdominal aortic aneurysm (AAA) devices (e.g., AAA stents, AAA grafts, etc.), filters (e.g., vena cava filters and mesh filters for distil protection devices), embolization devices including cerebral aneurysm filler coils (including Guglielmi detachable coils and metal coils), embolic agents, septal defect closure devices, drug depots that are adapted for placement in an artery for treatment of the portion of the artery distal to the device, myocardial plugs, pacemakers, leads including pacemaker leads, defibrillation leads and coils, neurostimulation leads such as spinal cord stimulation leads, deep brain stimulation leads, peripheral nerve stimulation leads, cochlear implant leads and retinal implant leads, ventricular assist devices including left ventricular assist hearts and pumps, total artificial hearts, shunts, valves including heart valves and vascular valves, anastomosis clips and rings, tissue bulking devices, suture anchors, tissue staples and ligating clips at surgical sites, cannulae, metal wire ligatures, tacks for ligament attachment and meniscal repair, joint prostheses, spinal discs and nuclei, orthopedic prosthesis such as bone grafts, bone plates, fins and fusion devices, orthopedic fixation devices such as interference screws in the ankle, knee, and hand areas, rods and pins for fracture fixation, screws and plates for craniomaxillofacial repair, dental implants, or other devices that are implanted or inserted into the body.

Surface materials may be selected, for example, from (a) organic materials (i.e., materials containing organic species, typically 50 wt % or more, for example, from 50 wt % to 75 wt % to 90 wt % to 95 wt % to 97.5 wt % to 99 wt % or more) such as polymeric materials (i.e., materials containing polymers, typically 50 wt % or more polymers, for example, from 50 wt % to 75 wt % to 90 wt % to 95 wt % to 97.5 wt % to 99 wt % or more) and biologics, (b) inorganic materials (i.e., materials containing inorganic species, typically 50 wt % or more, for example, from 50 wt % to 75 wt % to 90 wt % to 95 wt % to 97.5 wt % to 99 wt % or more), such as metallic inorganic materials (i.e., materials containing metals, typically 50 wt % or more, for example, from 50 wt % to 75 wt % to 90 wt % to 95 wt % to 97.5 wt % to 99 wt % or more) and non-metallic inorganic materials (i.e., materials containing non-metallic inorganic materials, typically 50 wt % or more, for example, from 50 wt % to 75 wt % to 90 wt % to 95 wt % to 97.5 wt % to 99 wt % or more), and (c) hybrid materials (e.g., hybrid organic-inorganic materials, for instance, polymer/metallic hybrids, polymer/ceramic hybrids, etc.).

Surface materials may be biostable or bioerodable.

Specific examples of metallic materials may be selected, for example, from biostable metals such as gold, iron, niobium, platinum, palladium, iridium, osmium, rhodium, titanium, tantalum, tungsten, ruthenium, zinc, and magnesium, among others, biostable alloys such as those comprising iron and chromium (e.g., stainless steels, including platinum-enriched radiopaque stainless steel), niobium alloys, titanium alloys, alloys comprising nickel and titanium (e.g., Nitinol), alloys comprising cobalt and chromium, including alloys that comprise cobalt and chromium (e.g., Elgiloy alloys), alloys comprising nickel, cobalt and chromium (e.g., MP 35N), alloys comprising cobalt, chromium, tungsten and nickel (e.g., L605), alloys comprising nickel and chromium (e.g., inconel alloys), bioerodable metals such as magnesium, zinc and iron, and bioerodable alloys including alloys of magnesium, zinc and/or iron (and their alloys with combinations of Ce, Ca, Al, Zr, La and Li), among others (e.g., alloys of magnesium including its alloys that comprises one or more of Fe, Ce, Al, Ca, Zn, Zr, La and Li, alloys of iron including its alloys that comprise one or more of Mg, Ce, Al, Ca, Zn, Zr, La and Li, alloys of zinc including its alloys that comprise one or more of Fe, Mg, Ce, Al, Ca, Zr, La and Li, etc.).

Specific examples of inorganic non-metallic materials may be selected, for example, from biostable and bioerodable materials containing one or more of the following: nitrides, carbides, borides, and oxides of various metals, including those above, among others, for example, aluminum oxides and transition metal oxides (e.g., oxides of iron, zinc, magnesium, titanium, zirconium, hafnium, tantalum, molybdenum, tungsten, rhenium, niobium, and iridium); silicon; silicon-based ceramics, such as those containing silicon nitrides, silicon carbides and silicon oxides (sometimes referred to as glass ceramics); various metal- and non-metal-phosphates, including calcium phosphate ceramics (e.g., hydroxyapatite); other bioceramics; calcium carbonate; carbon; and carbon-based, ceramic-like materials such as carbon nitrides.

Specific examples of organic materials include polymers (biostable or bioerodable) and other high molecular weight organic materials, and may be selected, for example, from suitable materials containing one or more of the following, among others: polycarboxylic acid homopolymers and copolymers including polyacrylic acid, alkyl acrylate and alkyl methacrylate homopolymers and copolymers, including poly(methyl methacrylate-b-n-butylacrylate-b-methyl methacrylate) and poly(styrene-b-n-butyl acrylate-b- styrene) triblock copolymers, polyamides including nylon 6,6, nylon 12, and polyether-block-polyamide copolymers (e.g., Pebax® resins), vinyl homopolymers and copolymers including polyvinyl alcohol, polyvinylpyrrolidone, polyvinyl halides such as polyvinyl chlorides and ethylene-vinyl acetate copolymers (EVA), vinyl aromatic homopolymers and copolymers such as polystyrene, styrene-maleic anhydride copolymers, vinyl aromatic-alkene copolymers including styrene-butadiene copolymers, styrene-ethylene-butylene copolymers (e.g., a poly(styrene-b-ethylene/butylene-b-styrene (SEBS) copolymer, available as Kraton® G series polymers), styrene-isoprene copolymers (e.g., poly(styrene-b-isoprene-b-styrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as poly(styrene-b-isobutylene-b-styrene) or SIBS, which is described, for instance, in U.S. Pat. No. 6,545,097 to Pinchuk et al.), ionomers, polyesters including polyethylene terephthalate and aliphatic polyesters such as homopolymers and copolymers of lactide (which includes d-,l- and meso-lactide), glycolide (glycolic acid) and epsilon-caprolactone, polycarbonates including trimethylene carbonate (and its alkyl derivatives), polyanhydrides, polyorthoesters, polyether homopolymers and copolymers including polyalkylene oxide polymers such as polyethylene oxide (PEO) and polyether ether ketones, polyolefin homopolymers and copolymers, including polyalkylenes such as polypropylene, polyethylene, polybutylenes (such as polybut-1-ene and polyisobutylene), polyolefin elastomers (e.g., santoprene) and ethylene propylene diene monomer (EPDM) rubbers, fluorinated homopolymers and copolymers, including polytetrafluoroethylene (PTFE), poly(tetrafluoroethylene-co-hexafluoropropene) (FEP), modified ethylene-tetrafluoroethylene copolymers (ETFE) and polyvinylidene fluoride (PVDF), silicone homopolymers and copolymers including polydimethylsiloxane, polyurethanes, biopolymers such as polypeptides, proteins, glycoproteins, polysaccharides, fibrin, fibrinogen, collagen, elastin, chitosan, gelatin, starch, and glycosaminoglycans such as hyaluronic acid; as well as blends and further copolymers of the above.

As noted above, due to the highly charged nature of the coatings the present disclosure, they are hydrophilic and thus suitable for use as lubricious coatings for medical devices.

Moreover, the coatings of the present disclosure are configured to self-destruct in physiological fluids over time. This is a desirable characteristic, particularly where the coatings are subjected to substantial mechanical stresses that can result in coating fragments becoming separated from the device, for example, where the devices are designed to traverse body lumens such as the coronary vasculature, peripheral vascular system, urinary tract, esophagus, stomach, intestines, colon, trachea, or biliary tract.

Many single use devices, such as catheters and wire interventional devices, among others, only require brief lubrication action during use. There is no requirement for a permanent lubricious coating and, in fact, such an approach may create additional questions or concerns during regulatory review. In addition to being self-destructive, the coatings of the present disclosure are also anti-thrombotic in some embodiments, making them particularly suitable for vascular applications.

In certain specific embodiments, the coatings of the present disclosure are applied to polymeric components of catheters, for example, the tubing and/or balloon components of angioplasty catheters. In this regard, materials used to form such components include polyamide materials. Examples of polyamide materials include nylon homopolymers and copolymers such as nylon 6, nylon 4/6, nylon 6/6, nylon 6/10, nylon 6/12, nylon 11 and nylon. Examples of polyamides further include polyether-polyamide block copolymers such as those containing (a) one or more polyether blocks selected from homopolymer and copolymer blocks containing one or more of ethylene oxide, trimethylene oxide, propylene oxide and tetramethylene oxide, (b) one or more polyamide blocks selected from nylon homopolymer and copolymer blocks such as nylon 6, nylon 4/6, nylon 6/6, nylon 6/10, nylon 6/12, nylon 11 and nylon 12 blocks.

A specific example of a polyether-polyamide block copolymer is poly(tetramethylene oxide)-nylon-12 block copolymer, available from Elf Atochem as Pebax®. Pebax® can be used to form tubing and balloons for angioplasty catheters, either alone or in combination with another material. As an example of the latter, the Mustang™ PTA Balloon Catheter from Boston Scientific Corporation is a 0.035 inch percutaneous transluminal angioplasty (PTA) catheter designed for a wide range of peripheral angioplasty procedures and employs Boston Scientific's NyBax™ Balloon Material, which is a co-extrusion of nylon and Pebax® polymers engineered to provide high-pressure, non-compliant dilatation in a low-profile balloon.

Pebax® materials are formed from lauryl lactam monomer and thus contain a residual amount of lauryl lactam. Unfortunately, lauryl lactam has been observed to migrate to the surface of Pebax® materials where it crystallizes to form particulates. Traditional hydrophilic coatings formed from non-ionic polymers such as polyethylene glycol (PEG) and polyvinyl pyrrolidone (PVP) can enhance the migration of lauryl lactam to the catheter surface and, after accumulation, its crystallization into particles.

Without wishing to be bound by theory, it is believed that the highly ionic hydrophilic coatings of the present disclosure, when coated on Pebax®, will result in a highly charged region at the surface of the Pebax® which is expected to discourage the migration of lauryl lactam to the surface, since the lauryl lactam molecule is less soluble in a high ionic strength environment.

Thus, the present disclosure describes hydrophilic polymer coatings that are lubricious and which, if fragmented and washed into the bloodstream, will bioerode for enhanced safety. The coatings may also discourage the formation of lauryl lactam surface particulates at Pebax® surfaces.

Although various embodiments are specifically illustrated and described herein, it will be appreciated that modifications and variations of the present invention are covered by the above teachings and are within the purview of the appended claims without departing from the spirit and intended scope of the invention.

1. A medical device having a negatively charged surface and a lubricous hydrophilic coating comprising a sulf(on)ated species disposed on the negatively charged surface, wherein the sulf(on)ated species is ionically crosslinked with itself and with the negatively charged entity by a multivalent cationic species.

2. The medical device of claim 1, wherein the medical device is a vascular catheter.

3. The medical device of claim 1, wherein the medical device comprises a polyether-block-polyamide copolymer component.

4. The medical device of claim 1, wherein the negatively charged surface comprises a surface-modified polyether-block-polyamide copolymer.

5. The medical device of claim 1, wherein the negatively charged surface is a negatively charged polymeric surface.

6. The medical device of claim 1, wherein the negatively charged polymeric surface comprises covalently attached negatively charged functional groups.

7. The medical device of claim 1, wherein the negatively charged polymeric surface comprises functional groups selected from carboxyl groups, sulfate groups, sufonate groups and combinations of the same.

8. The medical device of claim 1, wherein the negatively charged polymeric surface comprises covalently attached anionic small-molecules.

9. The medical device of claim 1, wherein the negatively charged polymeric surface comprises a covalently attached anionic polymer.

10. The medical device of claim 1, wherein the negatively charged polymeric surface comprises a conformally coated anionic polymer.

11. The medical device of claim 1, wherein the sulf(on)ated species comprise a polymer that comprises sulfate groups, sulfonate groups, or both.

12. The medical device of claim 1, wherein the sulf(on)ated species comprise a glycosaminoglycan.

13. The medical device of claim 1, wherein the sulf(on)ated species comprise heparin.

14. The medical device of claim 1, wherein the multivalent cationic species is a multivalent metal cation.

15. The medical device of claim 14, wherein the multivalent metal cation is selected from cations of magnesium, calcium, strontium, barium, iron, aluminum and zinc.

16. A medical device having a polymeric surface and a lubricous hydrophilic layer comprising a covalently crosslinked sulf(on)ated species disposed on the surface.

17. The medical device of claim 16, wherein the covalently crosslinked sulf(on)ated species comprises an ester-crosslinked sulf(on)ated species or wherein the covalently crosslinked sulf(on)ated species comprises an orthoester-crosslinked sulf(on)ated species.

18. The medical device of claim 16, wherein the sulf(on)ated species comprise a polymer that comprises sulfate groups, sulfonate groups, or both.

19. The medical device of claim 16, wherein the sulf(on)ated species comprise a glycosaminoglycan.

20. The medical device of claim 16, wherein the medical device is a vascular medical device.
